(19) United States
(12) Patent Application Publication
(10) Pub. No.: US 2026/0369351 A1
(43) Pub. Date: Nov. 19, 2026

(54) SYSTEM AND METHOD FOR REALISTIC MAMMALIAN DIGITAL TWIN

(71) Applicants: Vance Eugene Souders, II, Camden, NJ (US); Yijie Hu, Camden, NJ (US)

(72) Inventors: Vance Eugene Souders, II, Camden, NJ (US); Yijie Hu, Camden, NJ (US)

(21) Appl. No.: 19/641,023

(22) Filed: Apr. 7, 2026

Related U.S. Application Data
(60) Provisional application No. 63/806,176, filed on May 15, 2025.

(51) Int. Cl. G16B 20/20; C12Q 1/6869; G06N 3/042; G16B 30/00; G16B 40/00

(52) U.S. Cl. CPC G16B 20/20; C12Q 1/6869; G06N 3/042; G16B 30/00; G16B 40/00

(57) ABSTRACT

A system and method for generating a realistic mammalian digital twin (MDT) integrates genomic analysis and real-time biometric data to produce individualized and adaptive simulations of biological entities. The system performs DNA sequencing on a biological host and conducts single nucleotide polymorphism (SNP) analysis and/or DNA methylation analysis to derive genomically based behavioral phenotypes. A phenotype processor combines the behavioral phenotypes with optional real-time biometric data, such as heart rate, motion, and body temperature, transmitted via an encrypted wireless data transport layer, along with optional behavioral annotations, to produce a unified data stream. An AI behavior model applies a neurosymbolic approach to process the unified data stream together with a simulation world state and user input to generate behavioral outputs representing the mammalian digital twin. The system enables highly individualized and adaptive simulations suitable for applications including training, medical research, and digital therapeutics.

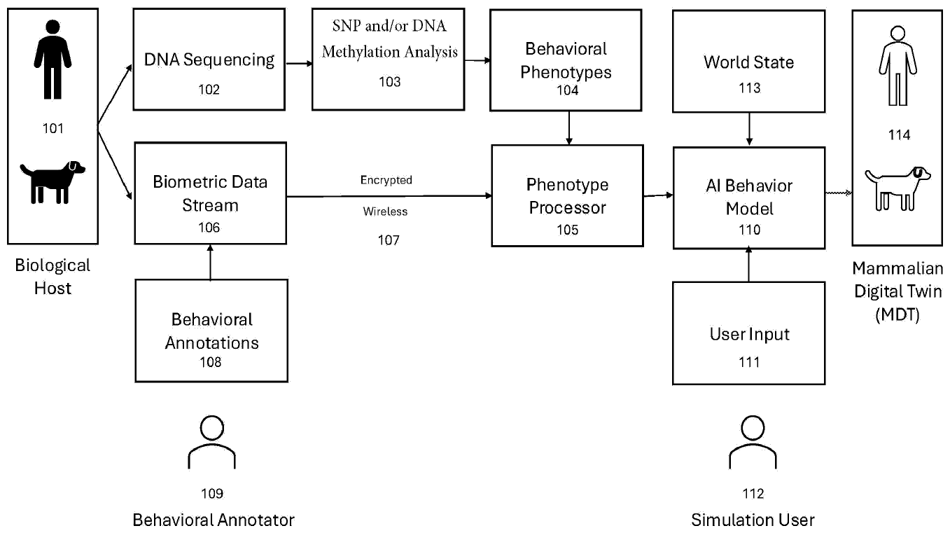

### CROSS-REFERENCE TO RELATED APPLICATION

This application claims priority to U.S. Provisional Patent Application No. 63/806,176, filed May 15, 2025, entitled “SYSTEM AND METHOD FOR REALISTIC MAMALIAN DIGITAL TWIN,” the entire disclosure of which is incorporated herein by reference.

### FIELD OF THE INVENTION

The present disclosure relates generally to biotechnology and computer simulation technologies, and more particularly to systems and methods for creating mammalian digital twins (MDTs) that integrate genomic analysis and real-time biometric data to produce highly individualized and adaptive simulations of biological entities.

### BACKGROUND OF THE INVENTION

The field of biotechnology and computer simulation has witnessed rapid advancements, driven by the need to model complex biological entities with increasing fidelity. A growing convergence of digital modeling techniques and biological data analysis has paved the way for simulating the behavior of living systems in controlled digital environments. This evolution is seen across industries where the translation of biological complexity into digital form is employed to explore and predict real-world outcomes. The integration of computational models with multidimensional biological information reflects a broader trend toward utilizing technology to emulate the intricate workings of living organisms.

In numerous applications, there is a recognized need for simulation methods that can act as effective substitutes for biological systems. Digital representations provide decision support in scenarios where traditional experimentation may be impractical, resource-intensive, or associated with notable safety risks. These applications range from training exercises under hazardous conditions to strategic planning in dynamic operational environments. By offering a virtual platform for assessment and analysis, such simulations aim to inform important decision-making and improve preparatory measures in situations that demand a detailed understanding of biological responses.

Despite these advances, existing simulation techniques often encounter limitations in fully capturing the complexity that is characteristic of biological systems. Many conventional approaches rely on static or narrowly scoped data, which may not account for the dynamic and multifaceted nature of living organisms. Without the integration of comprehensive biological markers or continuously updated physiological data, digital models may not accurately reflect complex behaviors. As a result, the predictive quality and reliability of such simulations can be compromised, particularly in applications where subtle variations in biological function are significant.

More specifically, there remains an unmet need in the art to incorporate a wider range of biological detail into digital simulations. Current methods may fall short by not sufficiently accounting for both genetic information and dynamic, real-time physiological signals. This omission can lead to simulated models that fail to reflect the distinguishing features of individual biological entities, potentially resulting in less reliable outcomes. There accordingly remains a need in the art for improved systems and methods for creating mammalian digital twins that achieve greater simulation fidelity by integrating genomic data and real-time biometric data.

### SUMMARY OF THE INVENTION

The present invention provides systems and methods for creating realistic mammalian digital twins (MDTs) through integration of genomic analysis, including single nucleotide polymorphism (SNP) analysis and/or DNA methylation analysis, with optional real-time biometric data streams, and artificial intelligence (AI) behavioral modeling.

In one aspect, the present invention provides a system for generating a mammalian digital twin, comprising: a genomic analysis module configured to perform DNA sequencing on a biological host and to conduct SNP and/or DNA methylation analysis to derive one or more behavioral phenotypes associated with the biological host; a phenotype processor configured to receive the one or more behavioral phenotypes and to integrate such phenotypes with real-time biometric data from a biometric data stream to produce a unified data stream; an AI behavior model configured to receive the unified data stream and to generate behavioral outputs representing actions and responses of a mammalian digital twin; and a simulation output representing the mammalian digital twin that dynamically adapts based on the unified data stream, a simulation world state, and user input.

In another aspect, the present invention provides a method for generating a mammalian digital twin, comprising: performing DNA sequencing on a biological host; analyzing the resulting genetic information to identify single nucleotide polymorphisms and/or DNA methylation patterns associated with one or more behavioral phenotypes; integrating the behavioral phenotypes with optional real-time biometric data to produce a unified data stream; and applying an AI behavior model to the unified data stream, in conjunction with a simulation world state and user input, to generate a mammalian digital twin.

In yet another aspect, the present invention provides a non-transitory computer-readable medium storing instructions that, when executed by one or more processors, cause a system to perform operations for generating a mammalian digital twin as described herein.

The system and method of the present invention provide a number of advantages over prior art approaches. The integration of genomically derived phenotypes and real-time biometric data enables the creation of highly individualized and responsive models. The modular architecture of the system supports scalability and adaptability across use cases ranging from simple embedded systems and robotics to fully immersive three-dimensional simulation environments.

These and other features, aspects and advantages of the present invention will become better understood with reference to the following drawings, description and claims.

### BRIEF DESCRIPTION OF THE DRAWINGS

The accompanying drawing, which is incorporated in and constitutes a part of this specification, illustrates an embodiment of the invention, and together with the description, serves to explain the principles of the invention. In the drawing:

The Sole Figure is a schematic block diagram illustrating a system architecture for creating a Mammalian Digital Twin (MDT) using genomic and biometric data, according to an embodiment of the present disclosure. The figure depicts a biological host (101) whose DNA is subjected to DNA sequencing (102) and SNP/DNA methylation analysis (103) to derive behavioral phenotypes (104), which are processed by a phenotype processor (105) together with a biometric data stream (106) transmitted via an encrypted wireless data transport layer (107) and behavioral annotations (108) provided by a behavioral annotator (109). The phenotype processor (105) provides a unified data stream to an AI behavior model (110), which also receives user input (111) from a simulation user (112) and a simulation world state (113), and generates the mammalian digital twin (MDT) (114) as an output.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Referring now to the figure, a system architecture for creating a Mammalian Digital Twin (MDT) 114 is illustrated according to an embodiment of the present invention. The system integrates multiple components to simulate the behavior of a biological host 101 with high fidelity by leveraging DNA sequencing 102, SNP analysis and/or DNA methylation analysis 103, real-time biometric data streams 106, and an AI behavior model 110.

Biological Host (101). The biological host 101 represents the living entity whose behavior is to be simulated. The biological host 101 may include, without limitation, humans, canines, equines, or other mammals. The system is configured to generate a digital twin 114 that corresponds to the specific genetic and physiological characteristics of the biological host 101.

DNA Sequencing (102). DNA sequencing 102 is performed on the biological host 101 to extract genetic information specific to that host. In embodiments, DNA sequencing 102 is performed using one or more standard genetic analysis tools or platforms. In embodiments, DNA sequencing 102 may be performed at regular intervals to incorporate changes in DNA methylation patterns that may occur over time, thereby allowing the system to remain current with the evolving epigenetic state of the biological host 101.

SNP and/or DNA Methylation Analysis (103). The genetic information derived from DNA sequencing 102 is subjected to SNP analysis and/or DNA methylation analysis 103. This analysis identifies single nucleotide polymorphisms and/or methylation patterns relevant to phenotypic traits of the biological host 101. Such traits may include, without limitation, athletic performance, cognitive abilities, disease susceptibility, and behavioral tendencies. The output of SNP and/or DNA methylation analysis 103 serves as a genomically derived foundational dataset for modeling the behavior of the biological host 101 in the simulation.

Behavioral Phenotypes (104). The SNP and/or DNA methylation analysis 103 maps identified polymorphisms and/or methylation patterns to one or more behavioral phenotypes 104. The behavioral phenotypes 104 represent quantified genomic traits that influence the simulated behavior of the mammalian digital twin 114. In embodiments, the behavioral phenotypes 104 are encoded within the phenotype processor 105 as a baseline behavioral dataset.

Phenotype Processor (105). The phenotype processor 105 integrates the genomically derived behavioral phenotypes 104 with optional real-time biometric data received from the biometric data stream 106, and optionally with behavioral annotations 108, to produce a unified data stream. The phenotype processor 105 serves as the central integration point for multi-modal biological data, providing a comprehensive and continuously updated representation of the biological host's physiological and behavioral state for use by the AI behavior model 110.

Biometric Data Stream (106). The biometric data stream 106 comprises physiological signals collected from the biological host 101 in real time or near-real time. Such physiological signals may include, without limitation, heart rate, motion data, body temperature, blood oxygen saturation (SpO2), and other measurable biometric parameters. The biometric data stream 106 enables the system to dynamically adapt the behavioral model of the mammalian digital twin 114 as the physiological state of the biological host 101 changes over time. The use of biometric data is optional; in embodiments where real-time biometric data is not available, the system may operate based on genomic data and behavioral annotations alone.

Data Transport Layer (107). The biometric data stream 106 is transmitted to the phenotype processor 105 via a data transport layer 107. In embodiments, the data transport layer 107 provides wireless and encrypted communication to ensure the secure transmission of biometric data. The data transport layer 107 may implement one or more wireless communication protocols, including but not limited to Bluetooth, Wi-Fi, Zigbee, Wi-Fi Direct, near-field communications (NFC), cellular network protocols (including GSM, CDMA, LTE), and the like.

Behavioral Annotations (108) and Behavioral Annotator (109). Behavioral annotations 108 represent observations, assessments, or classifications of the biological host's behavior derived from one or more data sources. In some embodiments, behavioral annotations 108 comprise manually entered data provided by a behavioral annotator 109, such as a trained human observer, clinician, or researcher. In other embodiments, the behavioral annotator 109 comprises an automated behavioral analysis module configured to generate behavioral annotations 108 using computer vision, machine learning algorithms, or pattern recognition applied to raw video, audio, or motion data. In certain embodiments, the behavioral annotator 109 operates in a hybrid configuration, wherein an AI-driven module generates candidate annotations that are subsequently validated or modified by a human user. These annotations supplement genomic and biometric data, providing critical ground-truth context to the AI behavior model 110, particularly in operational environments where direct biometric streaming may be intermittent or degraded.

AI Behavior Model (110). The AI behavior model 110 receives the unified data stream from the phenotype processor 105 and integrates this information with the simulation world state 113 and user input 111 to determine the behavior of the mammalian digital twin 114. In embodiments, the AI behavior model 110 employs a neurosymbolic approach, combining neural network-based learning with symbolic reasoning, to simulate the actions and responses of the mammalian digital twin 114 based on the combined data inputs. The AI behavior model 110 is configured to dynamically update the behavior of the mammalian digital twin 114 in response to changes in any of the input data sources.

User Input (111) and Simulation User (112). User input 111 is provided by a simulation user 112, who may interact with the mammalian digital twin 114 or the simulation environment. User input 111 may include, without limitation, instructions, interventions, or environmental changes introduced by the simulation user 112 into the simulation. Examples of user input 111 include providing simulated food or water to the digital twin, altering environmental conditions, or directing the digital twin to perform specific actions.

Simulation World State (113). The simulation world state 113 represents the environmental conditions and context within the simulation at any given point in time. The simulation world state 113 may include, without limitation, temperature, humidity, terrain, time elapsed, presence of other simulated entities, and other relevant environmental parameters. The AI behavior model 110 continuously updates the behavior of the mammalian digital twin 114 based on changes to the simulation world state 113. The simulation world state 113 represents the environmental conditions and context within the simulation at any given point in time. In Mixed Reality (MR) or Augmented Reality (AR) embodiments, the simulation world state 113 may be derived from, or synchronized with, real-world sensor data and telemetry. The simulation world state 113 may include, without limitation, temperature, humidity, terrain, time elapsed, presence of other simulated entities, physical obstacles in a real-world environment, and other relevant environmental parameters.

Mammalian Digital Twin (114). The mammalian digital twin 114 represents the concluding output of the system and serves as a virtual replica of the biological host 101. The mammalian digital twin 114 dynamically adapts its behavior in response to changes in the simulation world state 113, user interactions 111, and real-time biometric data stream 106, producing a realistic and personalized simulation. The mammalian digital twin 114 may be presented in various forms, including two-dimensional graphical representations, three-dimensional avatars in immersive simulation environments, abstract behavioral models for embedded systems or robotics platforms, and the like.

Exemplary Embodiment—Canine Digital Twin. In an exemplary embodiment, the biological host 101 is a canine. SNP and/or DNA methylation analysis 103 reveals a polymorphism within the ACE gene associated with enhanced endurance, which is encoded as a behavioral phenotype 104 within the phenotype processor 105. The phenotype processor 105 also receives biometric data from the canine host 101 over multiple sessions, including heart rate, motion data, body temperature, and environmental temperature. This combined data stream is provided to the AI behavior model 110, which computes an endurance factor for the canine mammalian digital twin 114 that accounts for both observed biometric data and genomic data as they relate to performance.

The AI behavior model 110 further refines the endurance factor based on the simulation world state 113; for example, the canine mammalian digital twin's 114 endurance may decrease in a simulated environment characterized by elevated temperature and high humidity. In a similar fashion, a simulation user 112 providing the mammalian digital twin 114 with simulated water may restore endurance.

Exemplary Use Case—Search and Rescue Training. In an exemplary use case, a team comprising five humans and three canine companions is scheduled to conduct search-and-rescue training in a game-based immersive simulation environment. Due to logistics, only three of the humans and none of the canine companions are available to participate in the exercise in person. The absent humans and all three canine companions are simulated using the mammalian digital twin system described herein.

SNP and/or DNA methylation analysis 103 indicates that two of the three canines (Canines A and B) have poor endurance, while the third (Canine C) has exceptional endurance. The AI behavior model 110 continuously processes changes in the simulated world state, driving the behavior of the simulated canines and humans. After three hours of simulated searching, Canines A and B become exhausted, as they were not provided with sufficient rest periods. As a result, the simulated mission is unable to continue and concludes in failure. This outcome illustrates that the incorporation of genetically derived phenotypic information has a direct and meaningful effect on the outcome of the simulation, and provides important lessons to the participants that may be applied in real-world settings.

Implementation. Embodiments of the invention and all of the functional operations described in this specification may be implemented in digital electronic circuitry, or in computer software, firmware, or hardware, including the structures disclosed in this specification and their structural equivalents, or in combinations of one or more of them. Embodiments may be implemented as one or more computer program products, e.g., one or more modules of computer program instructions encoded on a computer-readable medium for execution by, or to control the operation of, data processing apparatus. The computer-readable medium may be a non-transitory machine-readable storage device, a machine-readable storage substrate, or a memory device. The term “data processing apparatus” encompasses all apparatus, devices, and machines for processing data, including by way of example a programmable processor, a computer, or multiple processors or computers.

A computer program as used herein may be written in any form of programming language, including compiled or interpreted languages, and may be deployed in any form, including as a standalone program or as a module, component, subroutine, or other unit suitable for use in a computing environment. A computer program may be stored in a portion of a file that holds other programs or data, in a single file dedicated to the program, or in multiple coordinated files.

Processors suitable for execution of a computer program include, by way of example, both general and special purpose microprocessors, and any one or more processors of any kind of digital computer. Additionally, one or more non-von Neumann based architectures are also contemplated, such as memristor crossbar networks, quantum computers, and/or architectures based on the Harvard Architecture, Dataflow Architecture, Reduction Machines, Cellular Automata, and the like.

Embodiments of the invention may be implemented on a computing system that includes a back-end component (e.g., a data server), a middleware component (e.g., an application server), or a front-end component (e.g., a client computer having a graphical user interface or a web browser), or any combination of such components. The components of the system may be interconnected by any form or medium of digital data communication, including a local area network (LAN) or a wide area network (WAN) such as the Internet.

While this specification contains many specifics, these should not be construed as limitations on the scope of the invention or of what may be claimed, but rather as descriptions of features specific to particular embodiments. Certain features that are described in the context of separate embodiments may also be implemented in combination in a single embodiment. Conversely, various features described in the context of a single embodiment may also be implemented in multiple embodiments separately or in any suitable subcombination. Moreover, although features may be described above as acting in certain combinations, one or more features from a claimed combination may in some cases be excised from the combination.

It should be understood that the foregoing relates to exemplary embodiments of the invention and that modifications may be made without departing from the spirit and scope of the invention as set forth in the following claims.

What is claimed is:

1. A system for generating a mammalian digital twin, comprising:
a genomic analysis module configured to perform DNA sequencing on a biological host and to conduct at least one of single nucleotide polymorphism (SNP) analysis and DNA methylation analysis on genetic information derived from the DNA sequencing, to identify one or more behavioral phenotypes associated with the biological host;
a phenotype processor configured to receive the one or more behavioral phenotypes from the genomic analysis module and to integrate the one or more behavioral phenotypes with at least one of biometric data received from a biometric data stream and behavioral annotations, to produce a unified data stream; and
an AI behavior model configured to receive the unified data stream from the phenotype processor and to generate, based at least in part on the unified data stream, behavioral outputs representing actions and responses of a mammalian digital twin.

2. A method for generating a mammalian digital twin, comprising:
performing DNA sequencing on a biological host to obtain genetic information;
performing at least one of single nucleotide polymorphism (SNP) analysis and DNA methylation analysis on the genetic information to identify one or more behavioral phenotypes associated with the biological host;
integrating the one or more behavioral phenotypes with at least one of real-time biometric data from a biometric data stream and behavioral annotations to produce a unified data stream; and
applying an AI behavior model to the unified data stream to generate behavioral outputs representing actions and responses of a mammalian digital twin.

3. A non-transitory computer-readable medium storing instructions that, when executed by one or more processors, cause a system to perform operations comprising:
processing genetic information obtained from DNA sequencing of a biological host by performing at least one of single nucleotide polymorphism (SNP) analysis and DNA methylation analysis to identify one or more behavioral phenotypes associated with the biological host;
integrating the one or more behavioral phenotypes with at least one of biometric data received from a biometric data stream and behavioral annotations to produce a unified data stream; and
applying an AI behavior model to the unified data stream to generate behavioral outputs representing actions and responses of a mammalian digital twin.

4. The system of claim 1, wherein the biometric data stream comprises one or more of heart rate data, motion data, body temperature data, and blood oxygen saturation data collected from the biological host.

5. The system of claim 1, further comprising a data transport layer configured to transmit the biometric data stream to the phenotype processor via a wireless and encrypted communication channel.

6. The system of claim 1, wherein the AI behavior model is further configured to receive a simulation world state and user input, and to generate behavioral outputs based on the unified data stream, the simulation world state, and the user input.

7. The system of claim 6, wherein the simulation world state comprises one or more environmental parameters including at least one of ambient temperature, humidity, terrain, and elapsed simulation time.

8. The system of claim 1, wherein the AI behavior model employs a neurosymbolic approach combining neural network-based learning with symbolic reasoning.

9. The system of claim 1, wherein the biological host comprises at least one of a human, a canine, and an equine.

10. The system of claim 1, wherein the DNA sequencing is performed at regular intervals to incorporate changes in DNA methylation patterns of the biological host.

11. The system of claim 1, wherein the behavioral annotations are entered manually by a behavioral annotator.

12. The system of claim 1, wherein the mammalian digital twin is represented in at least one of a two-dimensional graphical representation, a three-dimensional avatar in an immersive simulation environment, and an abstract behavioral model for an embedded system or robotics platform.

13. The method of claim 2, further comprising transmitting the biometric data stream to a phenotype processor via a wireless and encrypted data transport layer.

14. The method of claim 2, further comprising receiving a simulation world state and user input, and generating the behavioral outputs based further on the simulation world state and the user input.

15. The method of claim 2, wherein applying the AI behavior model comprises employing a neurosymbolic approach combining neural network-based learning with symbolic reasoning.

16. The method of claim 2, wherein the one or more behavioral phenotypes comprise at least one of athletic performance, cognitive ability, and disease susceptibility.

17. The method of claim 2, wherein the biometric data stream comprises real-time physiological signals including at least one of heart rate, motion data, body temperature, and blood oxygen saturation.

18. The non-transitory computer-readable medium of claim 3, wherein the operations further comprise receiving a simulation world state and user input, and applying the AI behavior model to the unified data stream, the simulation world state, and the user input to generate the behavioral outputs.

19. The non-transitory computer-readable medium of claim 3, wherein the biometric data stream is received via a wireless and encrypted data transport layer.

20. The non-transitory computer-readable medium of claim 3, wherein the AI behavior model employs a neurosymbolic approach combining neural network-based learning with symbolic reasoning.
